(19) United States
(12) Patent Application Publication
(10) Pub. No.: US 2010/0280310 A1
(43) Pub. Date: Nov. 4, 2010

(54) Laparoscopic Gastric Band With Active Agents

(75) Inventor: Joseph S. Raven, Goleta, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/771,671

(22) Filed: Apr. 30, 2010

Related U.S. Application Data
(60) Provisional application No. 61/174,874, filed on May 1, 2009.

(51) Int. Cl. A61F 2/04; A61K 38/22; A61K 38/28

(52) U.S. Cl. 600/37; 514/12; 514/3

(57) ABSTRACT

A gastric banding system is provided which generally includes a gastric band and an active agent, for example, a metabolic agent or satiety inducing agent. The band may be structured to contain the agent and permit controlled release of the agent to the patient while the band is positioned around the stomach. Methods for treating obesity are also provided which include positioning a gastric band on the stomach of a patient and administering a satiety inducing agent to the patient while the gastric band is positioned on the stomach.

### RELATED APPLICATION

This application claims priority to U.S. Provisional Patent Application No. 61/174,874, filed on May 1, 2009, the entire disclosure of which is incorporated herein by this specific reference.

### BACKGROUND

The present invention relates to laparoscopic gastric banding for treatment of obesity and obesity related disorders and more specifically relates to a laparoscopic gastric band system including active agents.

Laparoscopic adjustable gastric bands have a successful history of inducing weight loss in obese patients. The band is secured around the stomach just below the gastroesophogeal junction. This creates a small pouch above the band which can only accept a small volume of food. Generally, this allows the patient to ingest only a small amount of food before the patient begins to feel satiated and full, and consequently, the patient is less likely to eat to excess. With reduced caloric intake, the patient loses weight. It is known that some patients, however, reach a “plateau” in their rate of weight loss over time, even with the gastric band in place.

Despite the relative safety and success of gastric banding in treating obesity and obesity related conditions, there remains a need for improved systems and methods for treating obesity and obesity related conditions in some patients.

### SUMMARY OF THE INVENTION

The present invention provides a gastric banding system generally comprising a gastric band structured to be placed around the stomach of a patient. Further, the band is capable of dispensing an active agent, for example, a metabolic agent, for example, but not limited to, a satiety inducing agent, to the patient while the band is positioned around the stomach. The system may provide more effective obesity treatment relative to obesity treatment using a gastric band alone.

For example, the system may further comprise a metabolic agent, or a satiety inducing agent, for being dispensed to the patient while the band is positioned around the stomach. The satiety inducing agent may be incorporated into the gastric band.

In one embodiment, an ancillary device is incorporated into the gastric band and the ancillary device includes, or is capable of dispensing to the patient, a satiety inducing agent. The ancillary device may be structured to provide controlled release of the satiety inducing agent to the patient.

For example, the ancillary device comprises a membrane or film permeable to a satiety inducing agent. The agent may be covered or enclosed by the membrane and is released into the body by diffusion through the membrane.

In other embodiments, the ancillary device may comprise a composition including a matrix material and a satiety inducing agent combined with the matrix material. The matrix material may be a bioerodible material, for example a bioerodible polymer which, during erosion thereof in the body, releases the agent from the composition in a controlled manner.

Alternatively, the ancillary device may be a non-bioerodible material. The device may include structures for containing and releasing the satiety inducing agents, for example in a controlled manner. In one embodiment, the ancillary device includes recessions, pores or grooves capable of containing a satiety inducing agent.

In some embodiments of the invention, the satiety inducing agent is a hormone, for example a peptide hormone. The peptide hormone may be at least one agent selected from the group consisting of Glucagon-like peptide (GLP-1), Oxyntomodulin (OXM), Peptide YY (PYY), Pancreatic Polypeptide (PP), Insulin, Leptin, Gastrin, Ghrelin blocker, an inhibitors of DPP-IV, and Amylin. The satiety inducing agent may be Cholecystokinin (CCK).

In other embodiments the ancillary device further includes a film or membrane in contact with the agent and capable of releasing the agent from the ancillary device and into the patient, for example, at a controlled rate.

In some embodiments, the gastric band itself is structured to be capable of releasing a satiety inducing agent into the patient at a controlled rate.

The present invention further provides a method of treating obesity or an obesity related condition in a patient. In one embodiment, the method comprises implanting a gastric band in a patient and providing a composition effective to induce satiety in the patient wherein the composition is positioned between the gastric band and the stomach of the patient when the gastric band is so positioned around the stomach of the patient.

For example, the composition may comprise composition as described elsewhere herein. For example, the composition may include a satiety inducing agent and a bioerodible material combined with the agent wherein the agent is distributed in the bioerodible material and is effective, when released into the patient, to at least assist in inducing satiety in the patient.

In another aspect of the invention, a method for treating obesity or an obesity related condition is provided wherein the method comprises positioning a gastric band on the stomach of a patient and administering a satiety inducing agent to the patient while the gastric band is positioned on the stomach.

The step of administering may comprise dispensing the agent to one of the stomach, intestine, peritoneum, intra-peritoneal cavity, and abdomen of the patient. In other embodiments, the agent is administered subcutaneously to the patient. In yet other embodiments, the step of administering comprises administering the agent directly to the central nervous system. In yet other embodiments, the agent is administered as an inhalant.

The step of administering may further comprise controlling a rate of release of the agent into the patient.

The agent may be administered at a controlled rate over a period of at least about six months, or at least about one year or at least about three years. In some embodiments, the controlled rate includes a period of dosage tapering, or a period of dosage increasing.

It is to be appreciated that the active agents useful in the present invention are not limited to satiety inducing agents but may also include any active agents, for example, other metabolic agents, that may provide some benefit to a patient suffering from obesity and/or obesity related conditions.

Each and every feature described herein, and each and every combination of two or more of such features, is included within the scope of the present invention provided that the features included in such a combination are not mutually inconsistent.

### BRIEF DESCRIPTION OF THE DRAWINGS

FIG. 1 is a perspective view of a system for treating obesity and obesity related conditions, in accordance with the invention.

FIGS. 2A and 2B are perspective views of surface structures useful for containing active agents in conjunction with a gastric band, in accordance with systems of the present invention.

FIG. 3 is a simplified representation of a diffusion material useful for controlling release of active agents in conjunction with a gastric band, in accordance with systems of the present invention.

### DETAILED DESCRIPTION

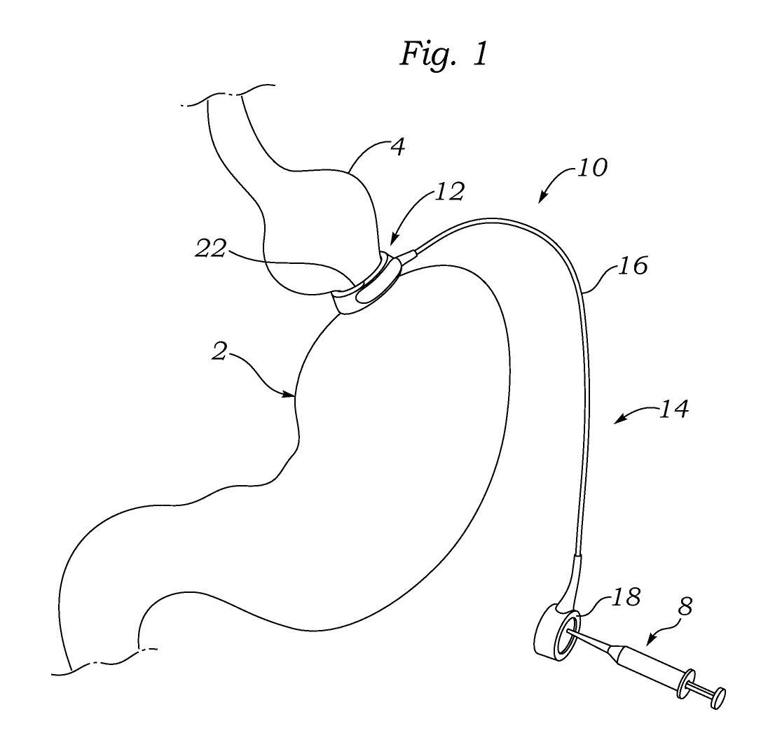

Turning now to FIG. 1, the present invention provides a gastric band system 10 which is structured to dispense an active agent, for example, a metabolic agent, for example a satiety inducing agent, for example, a satiety gut hormone or bioactive molecule, into the body. Although the present disclosure will typically be discussing, specifically, satiety inducing agents, it is to be appreciated that the present invention is not limited to active agents that are, specifically, satiety inducing agents. Active agents useful with the present invention are intended to include other compositions, drugs or other agents, for example, agents that affect body metabolism without necessarily affecting satiety, that are believed to be effective, at least to some degree, in facilitating weight loss in a human being.

In an exemplary embodiment, the system 10 generally comprises a gastric band 12 which is structured to be placed at the stomach 2 of a patient in such a manner so as to form a stoma 4, or pouch. The gastric band 12 may be an inflatable hydraulic gastric band (such as shown) or a mechanically adjustable gastric band. The gastric band 12 may include a stoma adjustment mechanism 14, comprising, for example, a fill line 16 and an implantable access port 18. By injecting or withdrawing a filling fluid from access port 18, for example, through the use of a needle/syringe 8, a physician can adjust a level of restriction of the band 12.

Further, the system 10 is capable of dispensing an active agent, for example, but not limited to, a satiety inducing agent, to the patient while the band is positioned around the stomach 2. The system 10 may provide more effective obesity treatment relative to obesity treatment using a gastric band alone.

For example, the system 10 may further comprise an active agent for being dispensed to the patient while the gastric band 12 is positioned at the stomach 2. The active agent may be incorporated into the gastric band.

In some embodiments, system further comprises an ancillary device 22 capable of dispensing to the patient, an active agent, while the system 10 is implanted in the patient. The ancillary device 22 may be incorporated into the gastric band 12, for example, at a region of the band 12 in contact with the stomach 2.

In some embodiments, the ancillary device comprises a composition incorporated into the gastric band. The composition may comprise a matrix material and an active agent, such as a satiety inducing agent, combined with the matrix material. The matrix material may be a bioerodible material, for example a bioerodible polymer which, during erosion thereof in the body, releases the agent from the composition, for example, in a controlled manner, for example in a time-release fashion.

Alternatively, the ancillary device may comprise a non-bioerodible material structured to facilitate release of an active agent into the body. In some embodiments, the device includes include structures for containing and releasing active agents, for example, in a controlled manner. Combinations of bioerodible and non-bioerodible materials for containing and releasing active agents are also contemplated.

In one embodiment, the ancillary device includes recessions, pores or grooves capable of containing an agent.

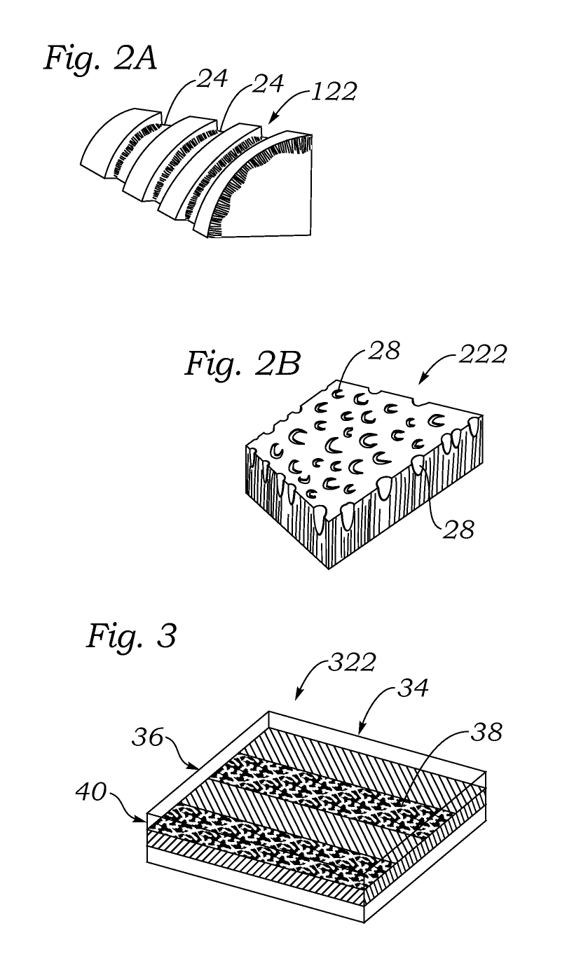

For example, an ancillary device 122, useful in the present systems, is shown FIG. 2A. Device 122 may include one or more of the features of ancillary device 22 described elsewhere herein.

Ancillary device 122 comprises a polymer surface having one or more indentations or grooves 24 capable of containing or holding a satiety inducing agent, or a composition containing a satiety inducing agent, for example, in solid, gel, powder, paste or other form.

Turning now to FIG. 2B, alternatively or additionally, the ancillary device 222 comprises a polymer surface having a porous or other irregular structure, wherein pores 28 are capable of containing or holding an agent, or a composition such as a matrix material containing an agent.

Ancillary device 22, 122, 222 may be made of any suitable, biocompatible material, for example, any suitable material approved by the Food and Drug Administration (FDA) for use in humans, for example, as approved for long term administration of agents. In one embodiment, the material is ethylene vinyl acetate (EVA).

In some embodiments, the active agent is a satiety inducing agent, for example, a hormone, for example a peptide hormone. The peptide hormone may be at least one agent selected from the group consisting of Glucagon-like peptide (GLP-1), Oxyntomodulin (OXM), Peptide YY (PYY), Pancreatic Polypeptide (PP), Insulin, Leptin, Gastrin, Ghrelin blocker, an inhibitors of DPP-IV, and Amylin. The satiety inducing agent may be Cholecystokinin (CCK).

In some embodiments of the invention, the active agent is an agent selected from a list of agents consisting of Glial-Derived Neurotrophic Factor (GDNF); Serotonin; Dopamine and its Analogues such as: Ibogaine, Noribogaine, 18-MC, and Cabergoline; Ciliary-derived Neurotrophic Factor (CNTF); Cocain-Amphetamine Regulated Transcript (CART); Serotonin and its Analogues; Gastric Inhibitory Peptide or Glucose-dependant Insulinotropic Peptide (GIP); Neuropeptide Y receptor antagonists and iRNA/siRNA; Orexin A receptor antagonists and iRNA/siRNA; Agouti Related Peptide (AgRP) receptor antagonists and iRNA/siRNA; Cannabanoid receptor antagonists and iRNA/siRNA; and the Melanocortins: Pro-Opiomelanocortin, Melanocyte Stimulating Hormone; Melanin Concentrating Hormone (MCH) receptor antagonists and iRNA/siRNA.

Discussions of gastrointestinal hormones that control appetite can be found in Chaudhri. O. B., Wynne, K., and Bloom, S. R. 2008. Can gut hormones control appetite and prevent obesity?. Diabetes Care 31 (Suppl. 2): s284-s289 and Cummings, D. E. and Overduin, J. 2007. Gastrointestinal regulation of food intake. J. Clin. Invest. 117: 13-23, the entire disclosures of which are incorporated herein by reference.

In other embodiments of the invention, the active agent may be any suitable active agent that will improve the weight-loss effect of the gastric band. For example, the active agent may be an agent that affects metabolism of a patient independently of the effect, if any, on satiety of the patient. Metabolic agents that are known or suspected to have a positive effect on weight loss are known to those of skill in the art.

Referring now as well to FIG. 3, in some embodiments of the invention, the ancillary device 22 comprises a film or membrane 322 which makes up a surface of the gastric band 12, for example, a surface of the band which contacts the stomach when the band is appropriately positioned. In one embodiment, the film 322 forms at least a portion of an inner circumferential surface of the gastric band 12. The film is capable of releasing a satiety inducing agent from the band and into the patient, for example, at a controlled rate.

For example, the film 322 may comprise a first membrane layer 34 and a second membrane layer 36. The film 322 may further comprise a composition containing a satiety inducing agent, wherein the composition is located adjacent, for example, between the first and second membrane layers 34, 36. The first and second membrane layers 34, 36 may comprise EVA or other suitable polymer or copolymer.

In the shown embodiment, the film 322 further comprises first and second agent layers 38, 40 which are made up of a composition containing a satiety inducing agent. The first and second agent layers 38, 40 are disposed in an alternating fashion with respect to the first and second membrane layers 34, 36. The membrane layers 34, 36 may have a known diffusion rate relative to the selected satiety inducing agent.

The film 322 is effective to control dosage and delivery of the agents to the patient. The film 322 may therefore have a desired porosity and/or be made of a suitable material so as to provide a controlled release of the agent.

For example, each of the ancillary devices described herein, for example, devices 122, 222 and 322, may be structured to provide effective concentrations of the agent for about six months, or for about one year, about two years, or about three years or more. In some embodiments, the devices 122, 222, 322 are structured to provide a sustained release rate, for example, of three years followed by a gradually decreasing release rate over the next about two to about three years. Numerous release protocols are contemplated by the inventors, and are understood to fall within the scope of the present invention.

The present invention further provides a method of treating obesity or an obesity related condition in a patient. In one embodiment, the method comprises implanting a gastric band in a patient and providing a composition effective to induce satiety in the patient wherein the composition is positioned between the gastric band and the stomach of the patient when the gastric band is so positioned around the stomach of the patient.

For example, the composition may comprise a composition as described elsewhere herein. For example, the composition may include a satiety inducing agent and a bioerodible material combined with the agent wherein the agent is distributed in the bioerodible material and is effective, when released into the patient, to at least assist in inducing satiety in the patient.

In another aspect of the invention, a method for treating obesity or an obesity related condition is provided wherein the method comprises positioning a gastric band on the stomach of a patient and administering a satiety inducing agent to the patient while the gastric band is positioned on the stomach.

The step of administering may comprise dispensing the agent to one of the stomach, intestine, peritoneum, intra-peritoneal cavity, and abdomen of the patient. In other embodiments, the agent is administered subcutaneously to the patient. In yet other embodiments, the step of administering comprises administering the agent directly to the central nervous system. In yet other embodiments, the agent is administered as an inhalant.

The step of administering may further comprise controlling a rate of release of the agent into the patient.

The agent may be administered at a controlled rate over a period of at least about six months, or at least about one year or at least about three years. In some embodiments, the controlled rate includes a period of dosage tapering, or a period of dosage increasing.

Exemplary peptide hormones which, alone or in combination, can be used in accordance with the invention include Glucagon-like peptide (GLP-1), Oxyntomodulin (OXM), Peptide YY (PYY), Pancreatic Polypeptide (PP), Amylin, Leptin, Gastrin or Ghrelin blocker. Another hormone that suppresses appetite when administered with or without gastric distension is Cholecystokinin (CCK), and other brain-gut satiety hormones such as Pro-opiomelanocortin (POMC).

In the publication, “Can Gut Hormones Control Appetite and Prevent Obesity?” by Chaudhri, et al, research conducted on Gherlin, GLP-1, Oxyntomodulin, Inhibitors of DPP-IV, Amylin, Peptide YY, and Pancreatic Polypeptide to control appetite, are described. These as well as other hormones may be useful in accordance with the present invention. Similarly, “Gastrointestinal Regulation of Food Intake” by David E. Cummings et al describes the efficacy of satiety hormones to boost weight loss.

The agent could also be applied to the band via a slow release drug eluting coating similar to coatings used on cardiovascular stents such as the Cordis Sirolimus Drug eluting stent or the contraceptive device Norplant. The coating could be applied directly to the band 12 for a slow release of the drug into the body.

An alternate method for dispensing the agent includes the provision of a semi-permeable membrane such as silicone or a nanostructure membrane as part of other implanted components of the system 10, for example, the fluid line 16 and/or access port 18. This would allow for a slow, for example, constant, diffusion of the agent into the body from other locations in the body.

### Example of GLP-1

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing glucagon like peptide 1 (GLP-1) that is released at a rate to achieve plasma concentrations of [10-30 pMol/L]ₚ GLP-1 over a period of 3-24 months. The patient reports a marked suppression of appetite, and within 12 months, the patient has lost 58 pounds.

### Example of OXM

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing oxyntomodulin (OXM) that is released at a rate to achieve plasma concentrations of [105-150 pMol/L]ₚ OXM over a period of 3-24 months. The patient reports a marked suppression of appetite, and within 12 months, the patient has lost 58 pounds.

### Example of PYY

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Peptide Y-Y (PYY) that is released at a rate to achieve plasma concentrations of [10-55 pMol/L]ₚ PYY over a period of 3-24 months. The patient reports a marked suppression of appetite, and within 12 months, the patient has lost 58 pounds.

### Example of PP

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Pancreatic Peptide (PP) that is released at a rate to achieve plasma concentrations of [150-300 pMol/L]ₚ PP over a period of 3-24 months. The patient reports a marked suppression of appetite, and within 12 months, the patient has lost 58 pounds.

### Example of Insulin

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Insulin that is released at a rate to achieve plasma concentrations of [5-30 μU/mL]ₚ Insulin over a period of 3-24 months. The patient reports a marked suppression of appetite, and within 12 months, the patient has lost 58 pounds.

### Example of Leptin

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Leptin that is released at a rate to achieve plasma concentrations of *[3-10 ng/mL]ₚ Leptin over a period of 3-24 months. The patient reports a marked suppression of appetite, and within 12 months, the patient has lost 58 pounds. *In the case of a female patient the goal plasma concentrations would be [10-20 ng/mL]ₚ.

### Example of Amylin

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Amylin that is released at a rate to achieve plasma concentrations of [20-25 pMol/L]ₚ Amylin over a period of 3-24 months. The patient reports a marked suppression of appetite, and within 12 months, the patient has lost 58 pounds.

### Example of CCK

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Cholecystokinin (CCK) that is released at a rate to achieve plasma concentrations of [5-10 pMol/L]ₚ CCK over a period of 3-24 months. The patient reports a marked suppression of appetite, and within 12 months, the patient has lost 58 pounds.

### Example of CNTF

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Ciliary neuro-trophic factor (CNTF) that is released at a rate to achieve plasma concentrations of [25-1300 pg/dL]ₚ CNTF over a period of 3-24 months. The patient reports a marked suppression of appetite, and within 12 months, the patient has lost 58 pounds.

### Example of CART

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing Cocaine-Amphetamine Regulated Transcript (CART) that is released at a rate to achieve plasma concentrations of [50-250 pM]ₚ CART over a period of 3-24 months. The patient reports a marked suppression of appetite, and within 12 months, the patient has lost 58 pounds.

### Example of Ghrelin Inhibition/Antagonism

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing a drug that is released at a rate to achieve plasma concentrations of Ghrelin at [15-30 pg/mL]ₚ over a period of 3-24 months. The patient reports a marked suppression of appetite, and within 12 months, the patient has lost 58 pounds.

### Example of NPY Inhibition/Antagonism

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing a drug that is released at a rate to achieve plasma concentrations of Neuro-peptide Y (NPY) at [65-95 pMol/L]ₚ over a period of 3-24 months. The patient reports a marked suppression of appetite, and within 12 months, the patient has lost 58 pounds.

### Example of Orexin A Inhibition/Antagonism

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing a drug that is released at a rate to achieve plasma concentrations of Orexin A at [20-50 pg/mL]ₚ over a period of 3-24 months. The patient reports a marked suppression of appetite, and within 12 months, the patient has lost 58 pounds.

### Example of AgRP Inhibition/Antagonism

A 49 year old male patient, having a body weight of 322 pounds and a height of 5′11″, complains to his physician that he has tried unsuccessfully to lose weight over the past 15 years and is concerned about the effect his excess weight may have on his health. At the physician's directive, the patient undergoes laparoscopic gastric banding surgery and has implanted around the upper part of his stomach a gastric band having a porous stomach-contacting surface, or a gastric band having a slowly drug eluting membrane, or a gastric band having a dissolvable film, or a gastric band with small grooves, containing a drug that is released at a rate to achieve plasma concentrations of AgRP at [1-16 ng/dL]ₚ over a period of 3-24 months. The patient reports a marked suppression of appetite, and within 12 months, the patient has lost 58 pounds.

Each of the publications cited in this application is incorporated herein in its entirety by this specific reference.

Although the invention has been described and illustrated with a certain degree of particularity, it is to be understood that the present disclosure has been made only by way of example, and that numerous changes in the combination and arrangement of parts can be resorted to by those skilled in the art without departing from the scope of the invention, as hereinafter claimed.

What is claimed is:

1. A gastric banding system comprising:
a gastric band structured to be placed around the stomach of a patient and the band being structured to be capable of dispensing an active agent to the patient while the band is positioned around the stomach.

2. The system of claim 1 further comprising an active agent for being dispensed to the patient while the band is positioned around the stomach.

3. The system of claim 1 further comprising a satiety inducing agent incorporated into the gastric band.

4. The system of claim 3 wherein the satiety inducing agent is a hormone.

5. The system of claim 3 wherein the satiety inducing agent is a peptide hormone.

6. The system of claim 5 wherein the peptide hormone is an agent selected from the group consisting of Glucagon-like peptide (GLP-1), Oxyntomodulin (OXM), Peptide YY (PYY), Pancreatic Polypeptide (PP), Insulin, Leptin, Gastrin, Ghrelin blocker, an inhibitors of DPP-IV, and Amylin.

7. The system of claim 3 wherein the satiety inducing agent is Cholecystokinin (CCK).

8. The system of claim 1 further comprising an ancillary device incorporated into the gastric band and capable of dispensing an active agent to the patient.

9. The system of claim 8 wherein the ancillary device is structured to provide controlled release of an active agent to the patient.

10. The system of claim 8 wherein the ancillary device comprises a membrane or film permeable to an active agent.

11. The system of claim 8 wherein the ancillary device includes grooves capable of containing an active agent.

12. The system of claim 8 wherein the ancillary device includes pores capable of containing an active agent.

13. The system of claim 8 wherein the ancillary device includes an active agent.

14. The system of claim 13 wherein the active agent is a satiety inducing agent.

15. The system of claim 13 wherein the ancillary device further includes a film or membrane in contact with the agent and capable of releasing the agent from the ancillary device and into the patient.

16. The system of claim 8 wherein the ancillary device comprises a film or membrane capable of releasing a satiety inducing agent from the ancillary device and into the patient at a controlled rate.

17. The system of claim 8 wherein the ancillary device is structured to be capable of releasing a satiety inducing agent into the patient at a controlled rate.

18. The system of claim 1 wherein the band is structured to be capable of releasing a satiety inducing agent into the patient at a controlled rate.

19. A method of inducing weight loss in a patient, the method comprising
implanting a gastric band device in a patient; and
providing a composition, the composition comprising an active agent effective to induce weight loss and a bioerodible material combined with the agent, the agent being distributed in the bioerodible material and being effective, when released into the patient, to at least assist in inducing weight loss in the patient;
the composition being positioned between the gastric band and the stomach of the patient when the gastric band is positioned around the stomach of the patient.

20. The method of claim 18 wherein the agent is an agent selected from the group consisting of Glucagon-like peptide (GLP-1), Oxyntomodulin (OXM), Peptide YY (PYY), Pancreatic Polypeptide (PP), Insulin, Leptin, Gastrin, Ghrelin blocker, an inhibitors of DPP-IV, and Amylin.

21. The method of claim 18 wherein the bioerodible material is capable of releasing the agent into the patient at a controlled rate.

22. A method of treating obesity or an obesity related condition comprising:
positioning a gastric band on the stomach of a patient; and
administering a satiety inducing agent to the patient while the gastric band is positioned on the stomach.

23. The method of claim 22 wherein the step of administering comprises dispensing the agent to one of the stomach, intestine, peritoneum, intra-peritoneal cavity, and abdomen of the patient.

24. The method of claim 22 wherein the step of administering comprises administering the agent subcutaneously to the patient.

25. The method of claim 22 wherein the step of administering comprises administering the agent directly to the central nervous system.

26. The method of claim 22 wherein the agent is administered as an inhalant.

27. The method of claim 22 wherein the step of administering comprises controlling a rate of release of the agent into the patient.

28. The method of claim 22 wherein the agent is selected from the group consisting of Glucagon-like peptide (GLP-1), Oxyntomodulin (OXM), Peptide YY (PYY), Pancreatic Polypeptide (PP), Insulin, Leptin, Gastrin, Ghrelin blocker, an inhibitors of DPP-IV, and Amylin.

29. The method of claim 22 wherein the step of administering comprises administering the agent at a controlled rate over a period of at least about six months.

30. The method of claim 22 wherein the step of administering comprises administering the agent at a controlled rate over a period of at least about one year.

31. The method of claim 22 wherein the step of administering comprises administering the agent at a controlled rate over a period of at least about three years.

32. The method of claim 22 wherein the step of administering comprises administering the agent at a controlled rate over a period of between about six months and about three years, the controlled rate including a period of dosage tapering.
